(19) United States
(12) Patent Application Publication
(10) Pub. No.: US 2022/0023590 A1
(43) Pub. Date: Jan. 27, 2022

(54) Mechanical Detachment Systems

(71) Applicant: RC Medical, LLC, Lake Forest, CA (US)

(72) Inventors: Brendon Bolos, Oceanside, CA (US); Hung Tien Nguyen, Placentia, CA (US); Samairot Louek, Santa Ana, CA (US); Erik Mulchandani, Irvine, CA (US); Randall Takahashi, Mission Viejo, CA (US)

(21) Appl. No.: 17/384,726

(22) Filed: Jul. 24, 2021

Related U.S. Application Data
(60) Provisional application No. 63/056,526, filed on Jul. 24, 2020.

(51) Int. Cl. A61M 25/01; A61M 25/00

(52) U.S. Cl. CPC A61M 25/0102; A61M 2025/0098; A61M 25/0053

(57) ABSTRACT

Mechanical Detachment Systems stemming from a corewire having a grooved slot for housing supplemental detachment means including SR threads, locking rings and variated pitches in helical groove driven systems which reliably detach instantaneously.

This application claims the full Paris Convention benefit of, and priority to, U.S. Provisional Patent Application Ser. No. 63/056,526, filed Jul. 24, 2020.

### FIELD OF THE INVENTION

The present invention relates to medical device detachment systems. Particularly neuro-vascular systems, for example, for delivery of brain coils.

### DETAILED DESCRIPTIONS

Mechanical Detachment Intellectual Property:

Groove Corewire with Mating Lock Ring;

A channel or groove is cut into the distal tip of the core-wire (˜1-1.5 mm length of groove);

The channel has a loose (0.6″ or greater) or tight (1.5 mm″ or less) pitch that winds in a helical pattern over the corewire tip outer diameter;

The channel may also have a zero (0) pitch;

A mating “lock ring” is part of or bonded to the main delivery catheter, ideally between the distal tip and the distal section of the delivery catheter;

The lock ring has a “nipple” feature, such that the “nipple” feature slides into and along the channel/groove, allowing the corewire to move freely/slide;

The “nipple” feature of the lock ring may be different shapes and sizes (e.g. rectangular or half-circle) to facilitate smooth sliding;

The lock ring is bonded and part of the delivery catheter, ideally onto the distal section, with the corewire being separate from the moving part, which moving part slides/pulled back for delivery of implant;

The mating lock ring may also not be bonded to the outer delivery catheter, but instead be part of a separate inner catheter;

The implant proximal end is placed/fitted into the channel or groove of the corewire;

The corewire is pulled back until the lock ring is flush with the distal end of the corewire, the implant proximal end engages the “nipple” of the lock ring, allowing the implant to be delivered.

The present inventors have overcome premature detachment issues in the market for example, Axiom®, 3Rivers® products, by adding a backbone design. The drawings and claims show the features.

### BRIEF DESCRIPTION OF THE DRAWINGS

FIG. 1 is a schematic illustration of a mechanical delivery system of the present invention.

FIG. 2 is a schematic illustration of mechanical delivery system of the present invention, showing movement of mating lock ring 103 from the first position along groove 105 of grooved corewire 101.

FIG. 3 is a schematic illustration of mechanical delivery system of the present invention, showing movement of mating lock ring 103 to a third position along groove 105 of grooved corewire 101.

FIG. 4 is a schematic illustration of a mechanical delivery system of the present invention, showing a partial plan view of grooved corewire 101 and mating lock ring 103.

FIG. 5 a schematic illustration of a mechanical delivery system of the present invention, which shows a rotated view of grooved corewire 101 and mating lock ring 103.

FIG. 6 is a schematic illustration of a mechanical delivery system of the present invention, which shows an implant 333 being pushed from the distal tip of the grooved corewire 101.

FIG. 7 is a schematic illustration of a mechanical delivery system of the present invention, which shows the nipple-like extension feature 269 in an extended position.

FIG. 8 is a schematic illustration of a mechanical delivery system of the present invention, which shows nipple-like extension feature 269 in the flush position. Mating lock ring 103 becomes flush with distal end of corewire 101, and implant 333 at proximal end engages the nipple-like extension 269 of mating lock ring 103, allowing the implant to be delivered.

FIG. 9 is a schematic illustration of a mechanical delivery system of the present invention, which shows the mating lock ring 103, showing that channel 105 is cut into the distal tip of said corewire 101.

FIG. 10 is a schematic illustration of a mechanical delivery system of the present invention showing the mating lock ring 103, bonded to an outer delivery catheter 202, with the nipple-like extension feature 269 in an extended position.

FIG. 11 is a schematic illustration of a mechanical delivery system of the present invention showing the mating lock ring 103, bonded to an outer delivery catheter 202, with the nipple-like extension feature 269 in a flush position.

FIG. 12 is a schematic illustration of a mechanical delivery system of the present invention showing an implant 333, ready for delivery by means of the instant delivery system.

FIG. 13 is a schematic illustration of a mechanical delivery system of the present invention showing the nipple-like extension feature 269 in an extended position.

FIG. 14 is a schematic illustration of a mechanical delivery system of the present invention showing the nipple-like extension feature 269 in a flush position.

FIG. 15 is a schematic illustration of a mechanical delivery system of the present invention showing the nipple-like extension feature 269 in a flush position, with an implant 333 ready for delivery.

FIG. 16 is a schematic illustration of a mechanical delivery system of the present invention showing nipple-like extension feature 269 in an extended position, with the implant 333 having just been delivered.

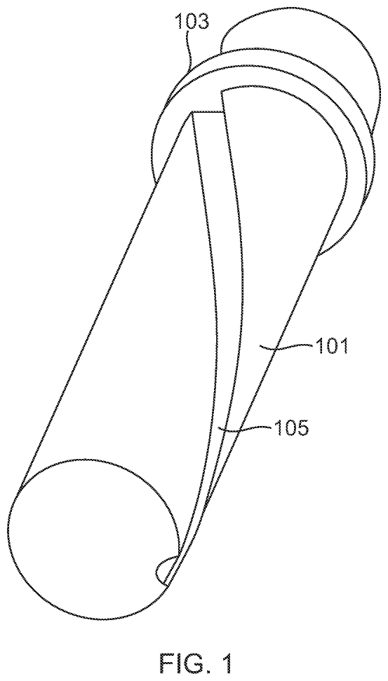

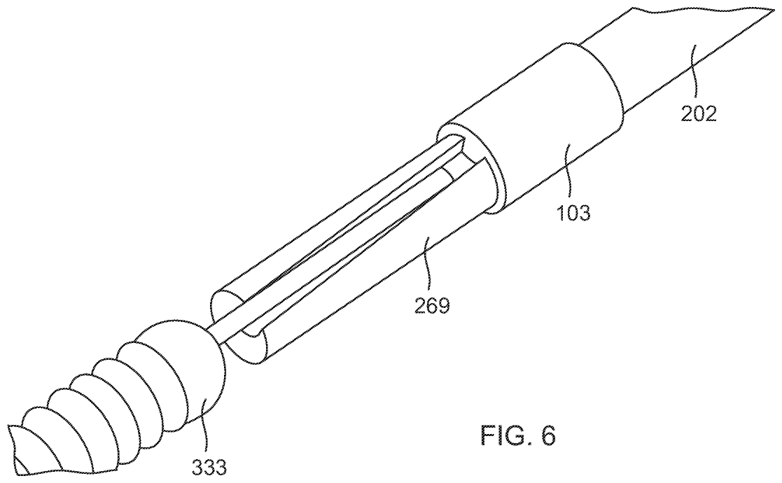

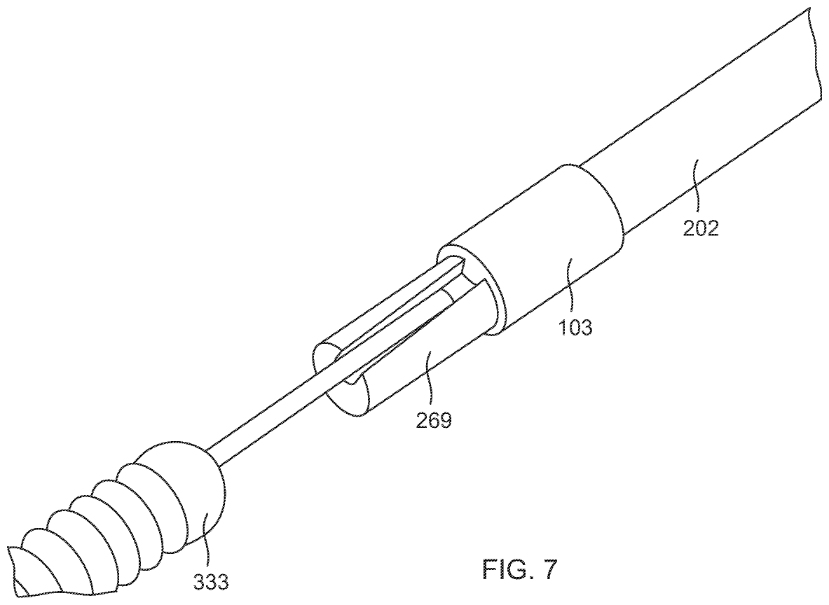

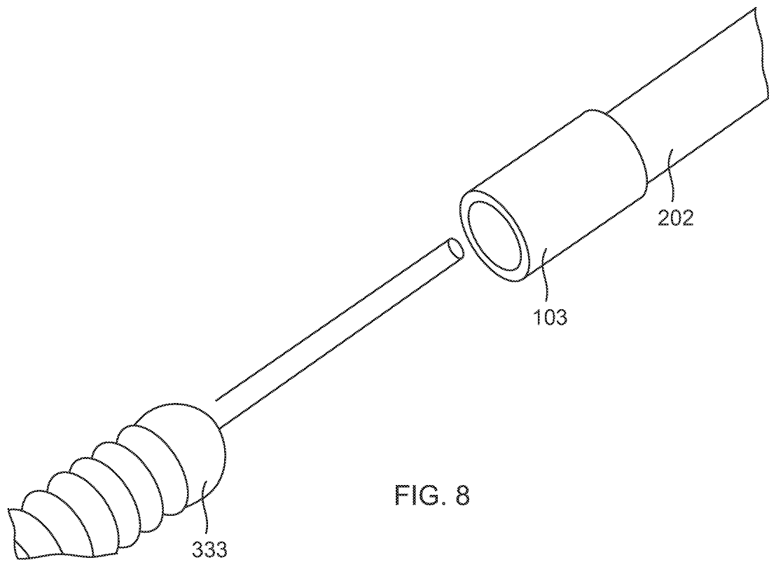

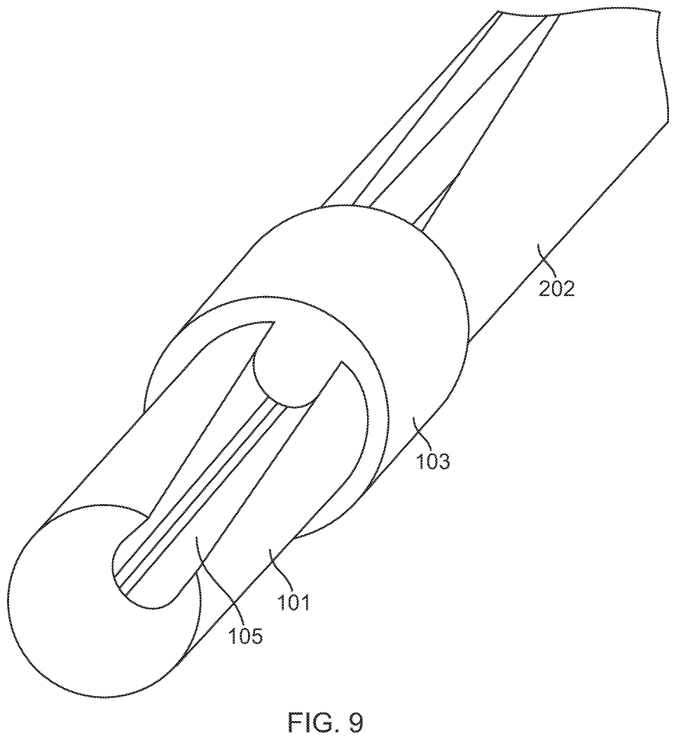

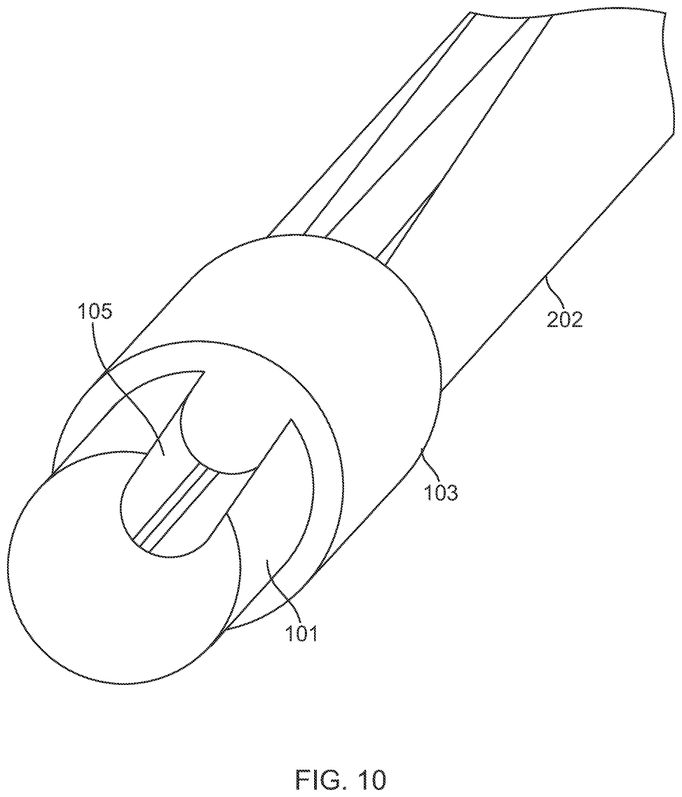

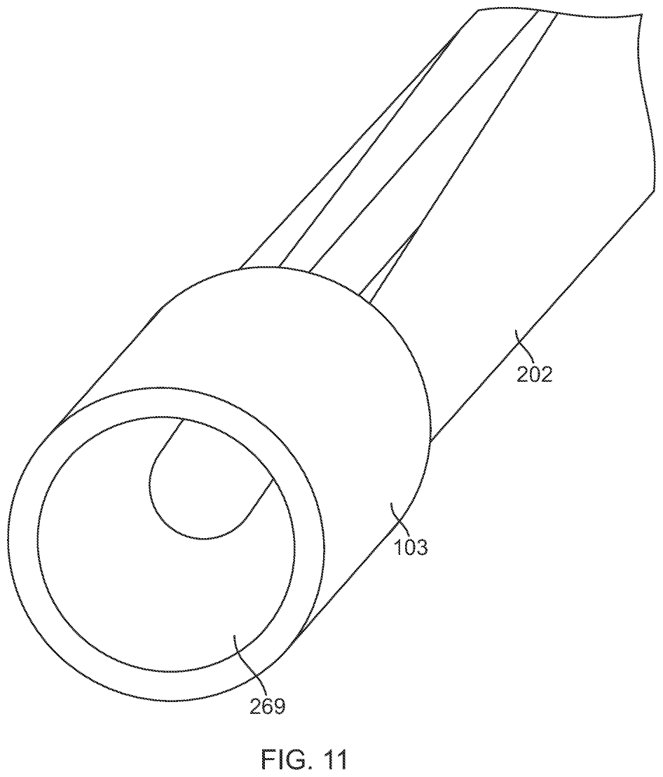

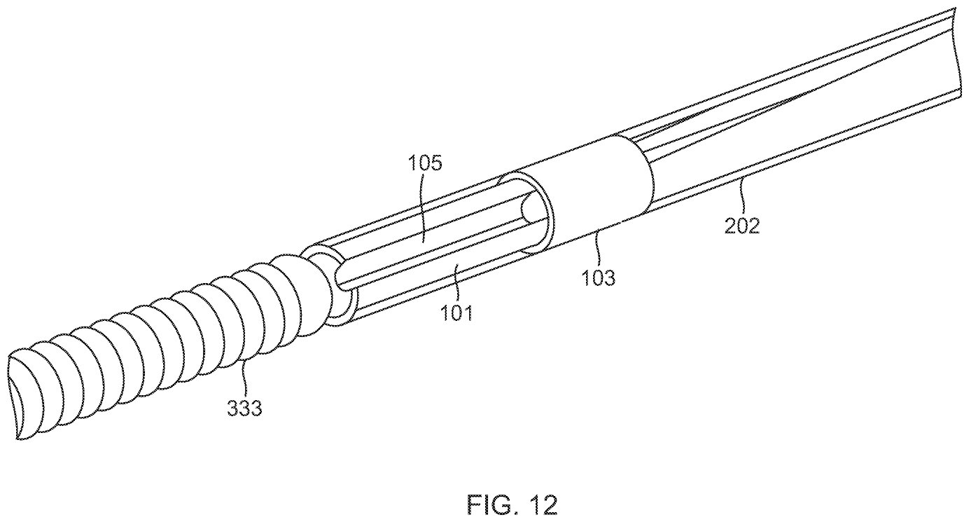

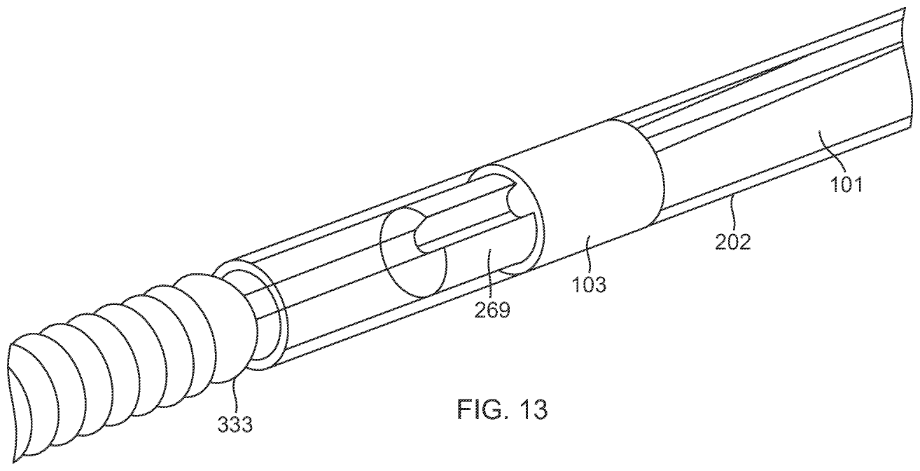

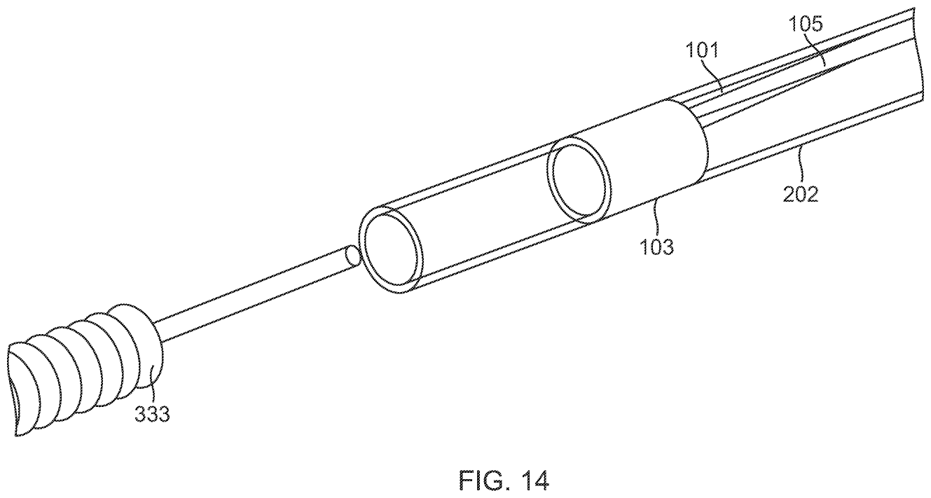

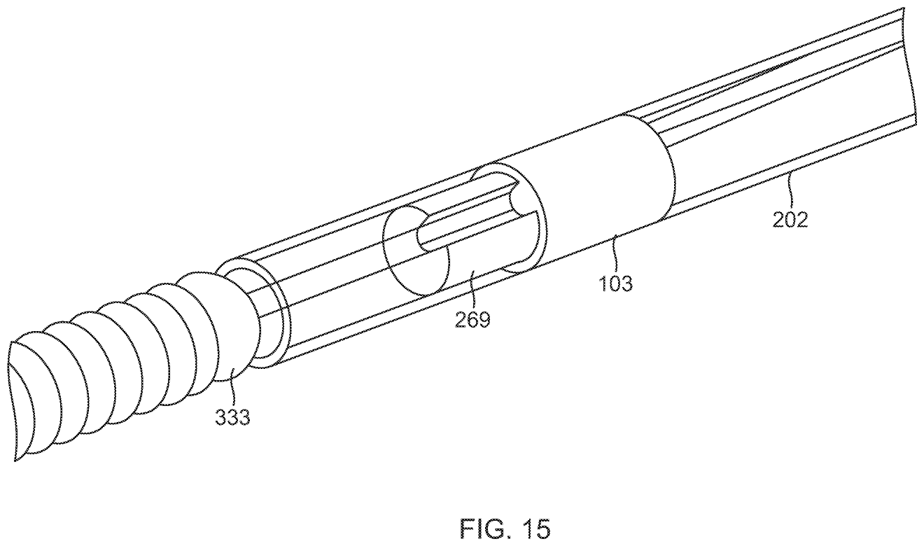

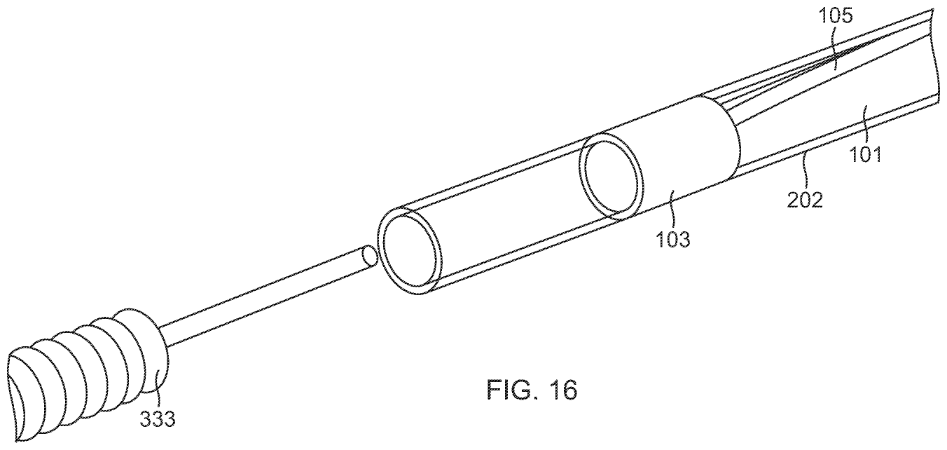

Referring now to FIGS. 1-16, and more particularly to FIGS. 9-16, the process for mechanical detachment of the instant system is informed by the methods of manufacturing to create desired features; namely, regarding grooved corewire 101, with mating lock ring 103, channel 105 is cut into the distal tip of said corewire 101 (for example, in illustrative but not limiting fashion, only between at least about 1 to 1.7 mm length of groove). Said channel further comprises a “loose” (again, for illustrative and not limiting purposes, around 0.6 mm or greater) or “tight” (approximately 1.5 mm or less in this example particularly shown) winding in a helical pattern over corewire 101 tip outer diameter (OD). Channel 105 may also have a zero pitch.

Referring now to FIG. 1, grooved corewire 101 includes mating lock ring 103 in a first position, disposed about groove 105.

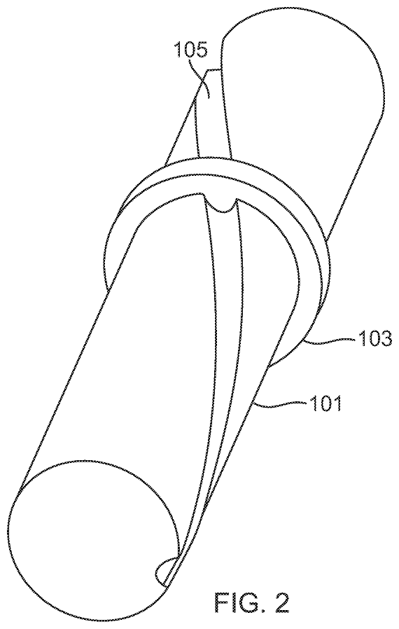

FIG. 2 shows movement of mating lock ring 103 from the first position along groove 105 of grooved corewire 101.

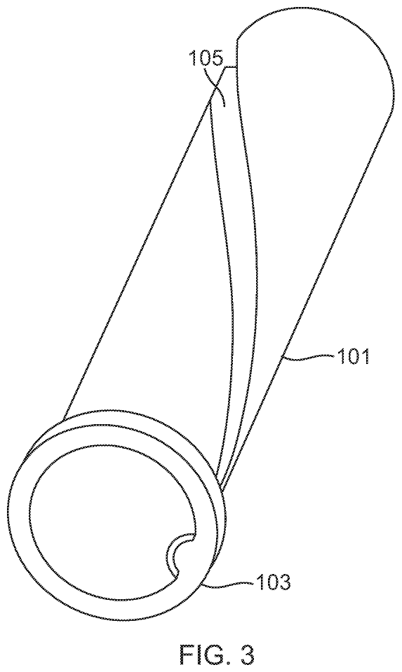

FIG. 3 shows movement of mating lock ring 103 to a third position along groove 105 of grooved corewire 101.

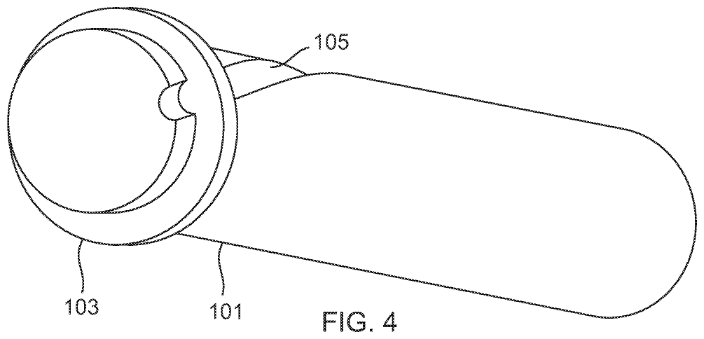

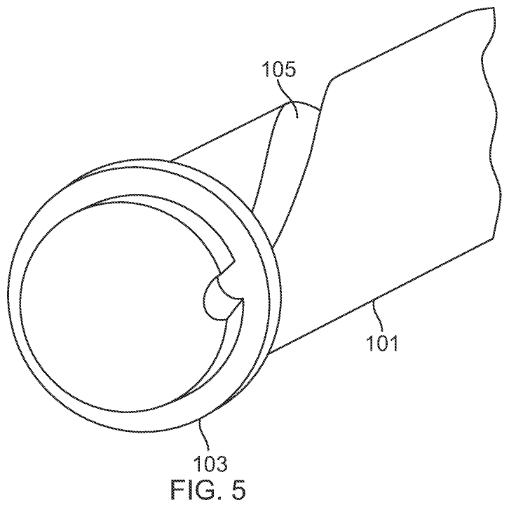

FIG. 4 shows a partial plan view of grooved corewire 101 and mating lock ring 103, while FIG. 5 shows a rotated view of the same.

Mating lock ring 103 is part of, or bonded to main delivery catheter 202, optimally between the distal tip and distal section of delivery catheter 202.

Mating lock ring 103 has a “nipple-like” extension feature 269, which may assume any number of geometric configurations, ranging from half-circle to rectangular-shaped to allow free movement of the corewire 101.

Mating lock ring 103 is bonded to and part of delivery catheter 202, ideally at the distal section, with corewire 101 being a separate moving part, sliding and pulling back for delivery of implant 333.

Mating lock ring 103 may also not be bonded to outer delivery catheter 202, but may be part of a separate inner catheter (not shown).

As corewire 101 is pulled back, mating lock ring 103 becomes flush with distal end of corewire 101, implant 333 at proximal end engages the nipple-like extension 269 of mating lock ring 103, allowing the implant to be delivered, as cartooned herein and claimed below.

Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially-feasible embodiment are often not depicted in order to facilitate a less obstructed view of these various embodiments of the present invention.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled. steps are indicative of one embodiment of the presented method. Other steps and methods may be conceived that are equivalent 111 function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed—some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using, consisting of, or consisting essentially of language. When used in the claims, whether as filed or added per amendment, the transition term “consisting of” excludes any element, step, or ingredient not specified in the claims. The transition term “consisting of” essentially limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the itivemion includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. Improved mechanical detachment systems, comprising, in combination:
a spiral core wire design with an HHS tube for stability, pushability and less friction, acting as a backbone design.

2. The improved mechanical detachment system of claim 1, further comprising:
a mating lock ring.

3. The improved mechanical detachment system of claim 2, further comprising:
at least a nipple-like feature for sliding into and along the at least a spiral grooved corewire design.

4. The improved mechanical detachment system of claim 3, where the mating lock ring is bonded to and part of the delivery catheter.

5. The improved mechanical detachment system of claim 4, the nipple extension feature having alternate geometric configurations.

6. The improved mechanical detachment system of claim 5, further comprising:
the mating lock ring not bonded to the outer catheter, but part of a separate inner catheter.

7. The improved mechanical detachment system of claim 6, further comprising:
an implant having a proximal end fitted with the channel of the corewire.

8. The improved mechanical detachment system of claim 7, whereby delivery is achieved when the corewire is pulled back until the lock ring is flush with the distal end of the corewire, and the implant proximal end engages the nipple-like extension of the lock ring.

9. The improved mechanical detachment system of claim 8, further comprising:
at least one of a loose and tight winding pitch for grooved corewire.

10. The improved mechanical detachment system of claim 9, further comprising a zero pitch channel.

11. Instant mechanical detachment systems, comprising, in combination:
a spiral grooved corewire element;
the spiral grooved corewire element functioning as a backbone by receiving other system members within its groove.

12. The improved mechanical detachment system of claim 11, further comprising a pitch for said groove.

13. The improved mechanical detachment system of claim 12, further comprising a loose winding pitch.

14. The improved mechanical detachment system of claim 11, further comprising a tight winding pitch.

15. The improved mechanical detachment system of claim 11, further comprising zero winding pitch.

16. The improved mechanical detachment system of claim 14, further comprising a mating lock ring.

17. The improved mechanical detachment system of claim 11, comprising at least a pin and strain relief (SR) thread.

18. The improved mechanical detachment system of claim 17, used to deliver neurological medical devices.

19. The improved mechanical detachment system of claim 12, said mechanical detachment system having at least one medical device of coils, flow diverters, and therapies.

20. The improved mechanical detachment system of claim 19, having no premature detachments.
